(19) United States
(12) Patent Application Publication
(10) Pub. No.: US 2024/0269158 A1
(43) Pub. Date: Aug. 15, 2024

(54) THERAPEUTIC AGENTS AND METHODS FOR TREATMENT OF BARTONELLOSIS

(71) Applicant: THE ADMINISTRATORS OF THE TULANE EDUCATIONAL FUND, New Orleans, LA (US)

(72) Inventor: Monica E. MORICI-EMBERS, Mandeville, LA (US)

(21) Appl. No.: 18/566,383

(22) PCT Filed: Jun. 2, 2022
(86) PCT No.: PCT/US22/31899

§ 371 Date: Dec. 1, 2023

Related U.S. Application Data
(60) Provisional application No. 63/195,907, filed on Jun. 2, 2021.

(51) Int. Cl. A61K 31/7052; A61K 31/431; A61P 31/04

(52) U.S. Cl. CPC A61K 31/7052; A61K 31/431; A61P 31/04

(57) ABSTRACT

Disclosed herein are therapeutic agents and combinations of therapeutic agents for use in the treatment of bacterial diseases. Such therapeutic agents and combinations may include azlocillin. Methods of reducing or inhibiting Bartonella bacteria in cells and/or in subjects having Bartonella infections and related disorders by providing therapeutic agents alone or in combination are provided.

### CROSS REFERENCE TO RELATED APPLICATIONS

This application claims priority to International PCT Application No. PCT/US22/31899, filed on Jun. 2, 2022, which claims the benefit under 35 U.S.C. § 119(e) to U.S. Provisional Application 63/195,907 filed on Jun. 2, 2021, entitled Therapeutic Agents and Methods for Treatment of Bartonellosis, the contents of which are herein incorporated by reference in their entirety.

### GOVERNMENT LICENSE RIGHTS

This disclosure was made with government support under grant 2P51OD011104-57 awarded by the National Institutes of Health. The government has certain rights in the disclosure.

### FIELD OF THE DISCLOSURE

The present disclosure relates to therapeutic agents, combinations and methods of treating a disease using the therapeutic agents. Specifically provided are therapeutic agents such as azlocillin for the treatment of diseases caused by bacteria.

### BACKGROUND

Bartonella bacteria occur worldwide and are the causative agents of multiple human diseases. The most well-known infection is called cat-scratch disease, which causes mild lymphadenopathy and fever. As the knowledge of these bacteria grow, new presentations of the disease with serious manifestations have been recognized. In addition to more severe disease, several new Bartonella species have been discovered in mammals. Considering the wide range of known clinical manifestations from the Bartonella infections, there remains a need to develop therapeutic strategies that target the causative bacteria, and which may be effective against all Bartonella-associated diseases.

### SUMMARY

The present disclosure provides methods related to the reduction in the levels of Bartonella bacteria in a cell or subject and/or methods of treating the diseases associated with Bartonella bacteria, e.g., Bartonellosis.

In some embodiments, the disclosure provides a method of reducing intracellular levels of Bartonella in a population of cells. Such methods include, contacting the population of cells with one or more therapeutic agents. In some embodiments, the therapeutic agent may be azlocillin, azithromycin, ceftriaxone, doxycycline, gentamicin, or ampicillin. Contacting the population of cells with one or more of therapeutic agents may reduce the intracellular levels of Bartonella in the population of cells. As a non-limiting example, the therapeutic agent may be azlocillin. A combination of two therapeutic agents may also be used to reduce the intracellular Bartonella levels. As a non-limiting example, the two therapeutic agents may be azlocillin and azithromycin. The concentration of azlocillin useful in the present disclosure may be from about 0.5 μg/ml to about 16 μg/ml. In some embodiments, the concentration of azlocillin may be from about 2 μg/ml to about 16 μg/ml. Further, the combination may include azithromycin at a concentration of from about 0.5 μg/ml to about 16 μg/ml. In some embodiments, the concentration of azithromycin may be from about 2 μg/ml to about 16 μg/ml.

The therapeutic agents described herein may be used to kill or inhibit the growth of Bartonella in a subject. Such methods may include, administering to the subject in need thereof with one or more therapeutic agents. In some embodiments, the therapeutic agent may be azlocillin, azithromycin, ceftriaxone, doxycycline, gentamicin, or ampicillin. The concentration of the therapeutic agent may be from about 0.001 μg/ml to about 5 μg/ml. In some embodiments, the therapeutic agent may be azlocillin. As a non-limiting example, the concentration of the therapeutic agent may be from about 0.001 μg/ml to about 2 μg/ml. In another aspect, the concentration of the azlocillin may be 0.25 μg/ml to about 2 μg/ml.

The present disclosure also provides methods of treating Bartonellosis in a subject. Such methods may include, administering to the subject in need thereof with one or more therapeutic agents. The therapeutic agent may be azlocillin, azithromycin, ceftriaxone, doxycycline, gentamicin, or ampicillin. In some embodiments, administering to the subject the therapeutic agent may reduce one or more symptoms associated with Bartonellosis. Bartonellosis may be caused by Bartonella bacilliformis, B. quintana, B. henselae, B. elizabethae, B. vinsonii, B. clarridgeiae, B. grahamii, B. koehlerae, B. alsatica, B. rochalimae, B. phoceensis, B. rattimassiliensis, B. tamiae, B. washoensis, or B. australis. In some embodiments, the Bartonellosis may be Cat Scratch Disease, Trench Fever, or Carrion's disease.

In Example 1, a method of reducing intracellular levels of Bartonella in a population of cells comprises contacting the population of cells with one or more therapeutic agents, wherein the one or more therapeutic agents is selected from the group consisting of azlocillin, azithromycin, ceftriaxone, doxycycline, gentamicin and ampicillin; and wherein contacting the population of cells with the one or more therapeutic agents reduces the intracellular levels of Bartonella in the population of cells.

Example 2 relates to the method according to Example 1, wherein the one or more therapeutic agents comprises azlocillin.

Example 3 relates to the method according to Example 1, wherein the method comprises contacting the population of cells with two therapeutic agents.

Example 4 relates to the method according to Example 3, wherein the two therapeutic agents are azlocillin and azithromycin.

Example 5 relates to the method according to Example 4, wherein the azlocillin is at a concentration of from about 0.5 μg/ml to about 16 μg/ml.

Example 6 relates to the method according to Example 4, wherein the azithromycin is at a concentration of from about 0.5 μg/ml to about 16 μg/ml.

Example 7 relates to the method according to Example 5, wherein the azlocillin is at a concentration of from about 2 μg/ml to about 16 μg/ml.

Example 8 relates to the method according to Example 6, wherein the azithromycin is at a concentration of from about 2 μg/ml to about 16 μg/ml.

In Example 9, a method of killing or reducing the growth of Bartonella in a subject comprises administering to the subject in need thereof one or more therapeutic agents, wherein the one or more therapeutic agents is azlocillin, azithromycin, ceftriaxone, doxycycline, gentamicin, or ampicillin; and wherein the administering to the subject in need thereof with the one or more therapeutic agents kills and/or inhibits the growth of the Bartonella.

Example 10 relates to the method according to Example 9, wherein the one or more therapeutic agents is at a concentration of from about 0.001 μg/ml to about 5 μg/ml.

Example 11 relates to the method according to Example 9 or 10, wherein the one or more therapeutic agents is azlocillin.

Example 12 relates to the method according to Example 11, wherein the azlocillin is at a concentration of from about 0.001 μg/ml to about 2 μg/ml.

Example 13 relates to the method according to Example 12, wherein the azlocillin is at a concentration of from about 0.25 μg/ml to about 2 μg/ml.

In Example 14, a method of treating Bartonellosis in a subject comprises administering to the subject in need thereof one or more therapeutic agents, wherein the one or more therapeutic agents is selected from the group consisting of azlocillin, azithromycin, ceftriaxone, doxycycline, gentamicin, and ampicillin; and wherein the administering to the subject in need thereof with the one or more therapeutic agents reduces one or more symptoms associated with Bartonellosis.

Example 15 relates to the method according to Example 14, wherein the Bartonellosis in the subject is caused by Bartonella bacilliformis, B. quintana, B. henselae, B. elizabethae, B. vinsonii, B. clarridgeiae, B. grahamii, B. koehlerae, B. alsatica, B. rochalimae, B. phoceensis, B. rattimassiliensis, B. tamiae, B. washoensis, or B. australis.

Example 16 relates to the method according to Example 14 or 15, wherein the Bartonellosis is Cat Scratch Disease, Trench Fever, or Carrion's disease.

Example 17 relates to the method according to any one of Examples 14-16, wherein the one or more therapeutic agents are independently at a concentration of from about 0.001 g/ml to about 5 μg/ml.

Example 18 relates to the method according to any one of Examples 14-16, wherein the method comprises administering two therapeutic agents to the subject in need thereof.

Example 19 relates to the method according to Example 18, wherein the two therapeutic agents are azlocillin and azithromycin.

Example 20 relates to the method according to Example 19, wherein the azlocillin is at a concentration of from about 0.5 μg/ml to about 16 μg/ml.

Example 21 relates to the method according to Example 20, wherein the azlocillin is at a concentration of from about 2 μg/ml to about 16 μg/ml.

Example 22 relates to the method according to Example 19, wherein the azithromycin is at a concentration of from about 0.5 g/ml to about 16 μg/ml.

Example 23 relates to the method according to Example 22, wherein the azithromycin is at a concentration of from about 2 μg/ml to about 16 μg/ml.

Example 24 relates to the method according to Example 14, wherein the one or more therapeutic agents is azlocillin.

Example 25 relates to the method according to Example 24, wherein the azlocillin is at a concentration of from about 0.001 μg/ml to about 2 μg/ml.

Example 26 relates to the method according to Example 25, wherein the azlocillin is at a concentration of from about 0.25 μg/ml to about 2 μg/ml.

While multiple embodiments are disclosed, still other embodiments will become apparent to those skilled in the art from the following detailed description, which shows and describes illustrative embodiments of the disclosure. Accordingly, the detailed description is to be regarded as illustrative in nature and not restrictive.

### DETAILED DESCRIPTION

Bartonellosis refers to a disease caused by one or more species of Bartonella. Three Bartonella species have been known to cause human disease and include Bartonella bacilliformis, Bartonella quintana, and Bartonella henselae. Among the different Bartonella species or subspecies that were recognized as causative agents of human diseases, these three species of Bartonella were reported to be responsible for the majority of clinical illnesses. Other Bartonella species known to cause diseases in humans include, but are not limited to, B. elizabethae, B. vinsonii, B. clarridgeiae, B. grahamii, B. koehlerae, B. alsatica, and B. rochalimae. In some embodiments, the Bartonella species may be B. phoceensis, B. rattimassiliensis, B. tamiae, B. washoensis, and/or B. australis. In some embodiments, the Bartonella species may be B. vinsonii subsp. arupensis, and/or B. vinsonii subsp. berkhoffii. The term “Bartonella ” (or “Bartonella spp.”) as used herein encompasses all Bartonella species or any one or more particular Bartonella species, unless the context clearly indicates otherwise.

The term “Bartonellosis” as used herein refers to any disease caused by Bartonella bacteria and may include Cat Scratch Disease (caused by Bartonella henselae), Trench Fever (caused by Bartonella quintana) and/or Carrion's disease (caused by Bartonella bacilliformis).

In humans, Bartonella infections have been associated with several clinical abnormalities that include arthralgia, arthritis, bacillary angiomatosis, endocarditis, myocarditis, cutaneous lesions, granulomatous hepatitis, neuroretinitis, peliosis hepatis, pulmonary nodules, uveitis, and vasoproliferative tumors (Cheslock M. A., et al. Trop Med Infect Dis 2019, 4:2, 69; the contents of which are herein incorporated by reference in its entirety). One study has shown a possible association between Bartonella infection and malignant melanoma (Ericson, M. E., et al. Pathogens 2021, 10, 326; the contents of which are herein incorporated by reference in its entirety). Using confocal microscopy techniques, Ericson, M. E., et al. have shown co-localization of B. henselae with vascular endothelial growth factor C (VEGFC), a melanoma growth factor in the skin biopsy tissues from the patients with melanoma. Another study focused on patients with neuropsychiatric disorders who reported concurrent cutaneous lesions (Breitschwerdt E B, et al. Pathogens. 2020 Dec. 4; 9(12): 1023; the contents of which are herein incorporated by reference in its entirety). Of these patients, 29/33 had positive serology or PCR for Bartonella.

Considering the wide range of known clinical manifestations from the Bartonella infections, there is no single treatment that effectively works against all Bartonella-associated diseases. Treatment regimens against Bartonella infections are primarily based on case reports that have limited number of patients, immunological status of the patient and/or symptoms of the patients. As a result, treatment has been confined to the immunological outcome of the disease rather than focusing on causative agent (Angelakis, E et al. Int J Antimicrob Agents 2014, 44, 16-25; the contents of which are herein incorporated by reference in its entirety). This suggests that a therapeutic strategy that may be effective in immunocompetent patients might not be suitable for a patient whose immune system is compromised. For example, gentamicin in combination with doxycycline is the treatment regimen recommended for human cases of Bartonella endocarditis, whereas ceftriaxone with gentamicin is recommended for infective endocarditis when Bartonella infection is suspected (Baddour et al. Circulation 2005, 111, e394-434; the contents of which are herein incorporated by reference in its entirety). For the treatment of Bartonella endocarditis in dogs and cats, doxycycline in conjunction with amikacin is recommended. Erythromycin, on the other hand, is the first-line antibiotic therapy for the treatment of patients with bacillary angiomatosis. Rifampicin or streptomycin have been used to treat verruga peruana and have been used to treat certain forms of Bartonellosis. Recent case reports presented hepatosplenic complications of cat-scratch disease (CSD) in immunocompetent individuals. Oral azithromycin is a proposed approach for the treatment of hepatosplenic CSD.

To avoid bacterial drug resistance, prolonged treatment periods must be avoided. New therapeutic combinations that are bactericidal, alongside therapeutic agents that could effectively penetrate the cell lipid barriers, considering intracellular nature of Bartonella, should be taken into account in the treatment of Bartonellosis. These therapeutic agents would also need to achieve concentrations within the cells for the effective killing of the bacteria. The present disclosure provides therapeutic agents and methods for treatment of Bartonellosis.

### Therapeutic Agents

The present disclosure provides therapeutic agents for the treatment of diseases such as, but not limited to, Bartonellosis. In some embodiments, treatment of the disease in a subject may involve administering a therapeutic agent to a subject. In some embodiments, the therapeutic agent may be azlocillin, azithromycin, ceftriaxone, doxycycline, gentamicin, ampicillin, or combinations thereof.

In some embodiments, the therapeutic agents may be useful in reducing or inhibiting the growth of early phase and/or stationary phase Bartonella.

In some embodiments, the therapeutic agent may be an antibiotic. As used herein, the term, “antibiotic” refers to any substance that inhibits the growth and replication of a bacterium and/or kills the bacterium, e.g., Bartonella. The antibiotic may be a beta lactam antibiotic, an aminoglycoside, a glycopeptide, an ansamycin, a streptogramin, a sulfonamide, a macrolide, an oxazolidinone, a peptidoglycan, a quinolone, and/or a lipopeptide.

### Azlocillin

In some embodiments, the therapeutic agent may be azlocillin. Azlocillin is an acylated form of ampicillin and is a broad range β-lactam that is similar to antibiotics such as mezlocillin and piperacillin.

In some embodiments, azlocillin may be administered at a concentration ranging from about 0.001 μg/ml to about 5 μg/ml. As a non-limiting example, the concentration of the therapeutic agent may be from about 0.001 μg/ml to about 2 μg/ml. In another aspect, the concentration of the azlocillin may be from about 0.25 μg/ml to about 2 μg/ml. As a non-limiting example, the concentration of the therapeutic agent may be from about 0.001 μg/ml to about 2 μg/ml. In another aspect, the concentration of the azlocillin may be from about 0.25 μg/ml to about 2 μg/ml, from about 1 μg/ml to about 50 μg/mL, from about 1 μg/mL to about 10 μg/mL; from about 5 μg/mL to about 15 μg/mL; from about 10 μg/mL to about 20 μg/mL; from about 15 μg/mL to about 25 μg/mL; from about 20 μg/mL to about 30 μg/mL; from about 25 g/mL to about 35 μg/mL; from about 30 μg/mL to about 40 μg/mL; from about 35 μg/mL to about 45 μg/mL; and/or from about 40 μg/mL to about 50 g/mL. The concentration of azlocillin may be from about 0.001 μg/ml to about 0.005 μg/ml, from about 0.005 μg/ml to about 0.01 μg/ml, from about 0.01 μg/ml to about 0.02 μg/ml, from about 0.02 to about 0.5 g/ml, from about 0.5 μg/ml to about 1.2 μg/ml, from about 1 μg/ml to about 2 μg/ml, from about 2 μg/ml to about 5 μg/ml, or from about 5 μg/ml to about 16 μg/ml. Various suitable dosages for administration of azlocillin to a subject (e.g., in amounts expressed in terms of μg/kg body weight to mg/kg body weight) are provided herein.

### Other Therapeutic Agents

In some embodiments, the therapeutic agent may be azithromycin.

In some embodiments, the therapeutic agents may be macrolides or tetracyclines. Inhibition in the growth of B. henselae in vitro was shown when tested with macrolides, tetracyclines, and rifampicin either using E-test methodology or the agar dilution method. Considering the good intracellular activity of these antibiotics, macrolides and tetracyclines have been used for the treatment of diseases caused by B. henselae (Tsuneoka, H et al. J Infect Chemother 2010, 16, 446-448; the contents of which are herein incorporated by reference in its entirety).

In some embodiments, the therapeutic agent may be minocycline. Minocycline is a tetracycline, which was shown to be effective at a concentration of <0.016 μg/ml against 32 isolates of B. henselae that were tested by E-test susceptibility (Tsuneoka, H et al. J Infect Chemother 2010, 16, 446-448). In some embodiments, the therapeutic agent may be rifampin, crythromycin, azithromycin, doxycycline, ciprofloxacin, or a combination thereof. One study demonstrated that rifampin, crythromycin, azithromycin, doxycycline, and ciprofloxacin antibiotics that are currently used to treat Bartonellosis show very poor activity against stationary phase B. henselae, but were effective against growing bacterial culture (Li, T. et al. Antibiotics (Basel) 2019, 8, the contents of which are herein incorporated by reference in its entirety). However, 6-day treatment with the antibiotic combination of azithromycin/ciprofloxacin, azithromycin/methylene blue, rifampin/ciprofloxacin, and rifampin/methylene blue was able to completely eradicate the growth of the bacteria in log and stationary phases (Feng, J. et al. PLOS One 2014, 9, e111809, doi: 10.1371/journal.pone.0111809; the contents of which are herein incorporated by reference in its entirety).

### Combinations

In some embodiments, a combination of therapeutic agents may be utilized. The present disclosure provides two, three, four, five or more therapeutic agents in a combinatorial format. Combinations may be administered concurrently, sequentially, and/or serially. In some embodiments, each therapeutic agent in a combination may be formulated as separate pharmaceutical formulations. In some embodiments, the therapeutic agents in a combination may be prepared as single pharmaceutical formulations.

For purposes of the present disclosure, “in combination,” refers to providing two or more therapeutic agents either separately or together, where the two therapeutic agents are administered as part of an appropriate dose regimen designed to obtain the benefit of the combination therapy. Thus, the two therapeutic agents can be administered either as part of the same pharmaceutical composition or in separate pharmaceutical compositions. The first therapeutic agent can be administered prior to, at the same time as, or subsequent to administration of the second therapeutic agent, or in some combination thereof. Where the one therapeutic agent is administered to the subject at repeated intervals, e.g., during a standard course of treatment, the second therapeutic agent can be administered prior to, at the same time as, or subsequent to, each administration of the first therapeutic agent, or some combination thereof, or at different intervals in relation to therapy with the first therapeutic agent, or in a single dose prior to, at any time during, or subsequent to the course of treatment with the first therapeutic agent.

Combinations of therapeutic agents of the present disclosure may include azlocillin and one or more other therapeutic agents. In some embodiments, azlocillin may be combined with a therapeutic agent such as, but not limited to, azithromycin, ceftriaxone, doxycycline, gentamicin or ampicillin. In some embodiments, the present disclosure provides a combination of therapeutic agents, such as, but not limited to, azlocillin and azithromycin. In other examples, the present disclosure provides a combination of azlocillin with other therapeutic agents, such as, but not limited to rifampin, erythromycin and/or doxycycline.

### Methods of Use

The present disclosure provides methods of use related to the therapeutic agents described herein. In some embodiments, the methods described herein may include a method of reducing the growth of bacteria such as Bartonella. Such methods may include contacting the bacteria with the therapeutic agent(s) of the disclosure.

In some embodiments, the present disclosure provides methods for inhibiting the growth of Bartonella bacteria. The Bartonella bacteria may be intracellular or extracellular. The growth of the Bartonella may be inhibited in vitro (e.g., in a cell or a tissue sample, e.g., from a subject) or in vivo in a subject. In some aspects, the growth of the Bartonella is inhibited within a cell or in a population of cells infected by the bacteria. In some embodiments, the growth of the bacteria is inhibited by about 10%, about 20%, about 30%, about 40%, about 50%, about 60%, about 70%, about 80% and/or about 90%. In some embodiments the growth of the bacteria is inhibited by between about 5-15%, about 10-20%, about 15-25%, about 20-30%, about 25-35%, about 30-40%, about 35-45%, about 40-50%, about 45-55%, about 50-60%, about 55-65%, about 60-70%, about 65-75%, about 70-80%, about 75-85%, and/or about 90-100%. In some embodiments, the Bartonella bacteria are killed upon treatment with the therapeutic agent.

### Bartonellosis

In some embodiments, the therapeutic agent(s) and combinations thereof of the disclosure may be used to treat Bartonellosis. Any disease caused by or associated with infection by one or more species of Bartonella is herein described as Bartonellosis. Three Bartonella species have been known to cause human disease and include Bartonella bacilliformis, Bartonella quintana, and Bartonella henselae. Among the different Bartonella species or subspecies that were recognized as causative agents of human diseases, these three species of Bartonella were reported to be responsible for the majority of clinical illnesses. Other Bartonella species known to cause diseases in humans include, but are not limited to, B. elizabethae, B. vinsonii, B. clarridgeiae, B. grahamii, B. koehlerae, B. alsatica, and B. rochalimae. In some embodiments, the Bartonella species may be B. phoceensis, B. rattimassiliensis, B. tamiae, B. washoensis, or B. australis. In some embodiments, the Bartonella species may be B. vinsonii subsp. arupensis, and/or B. vinsonii subsp. berkhoffii.

Therapeutic agents of the disclosure may be used to treat Bartonellosis. Bartonellosis may include Carrion's disease, trench fever, cat scratch disease, Bartonella endocarditis, among others.

In some embodiments, the subject infected with one or more Bartonella species may have an otherwise intact immune system. In some embodiments, the subject infected with one or more Bartonella species may be immunocompromised. Subjects who are immunocompromised may have a reduced ability to fight infections and other diseases. A compromised immune system may be caused by certain diseases or conditions, such as but not limited to, AIDS, cancer, diabetes, malnutrition, and certain genetic disorders. In some embodiments, the subject may be immune-compromised due to certain medicines or treatments, such as anticancer drugs, radiation therapy, and stem cell or organ transplant.

### Carrion's Disease

In some embodiments, the therapeutic agents of the disclosure may be used to treat Carrion's disease which may be caused by B. bacilliformis. This bacterium causes both an acute febrile illness (Carrion's disease or Oroya fever) and a chronic vasoproliferative disease (verruga peruana). In the acute illness, usually occurring within 21 days of an infected sandfly bite, there is a severe intravascular hemolytic anemia, with symptoms of high fever, anemia, and temporary immunosuppression, lasting from two to four weeks. In some embodiments, the therapeutic agents and combinations thereof may be used to treat the acute illness associated with Carrion's disease. B. bacilliformis may also invade endothelial cells causing nodular skin lesions to appear; these lesions can be pruritic and bleed spontaneously or if abraded. Immunosuppression, secondary to B. bacilliformis, resulting in tuberculosis and other coinfections, has also been suggested to occur in association with long-standing intravascular B. bacilliformis infection resulting in verruga peruana. Therapeutic agents of the disclosure and combinations thereof may be used to treat verruga peruana.

### Trench Fever

In some embodiments, the therapeutic agents of the disclosure may be used to treat trench fever which may be caused by B. quintana. The therapeutic agents of the disclosure may be used to treat acute (trench fever) and/or chronic (endocarditis and bacillary angiomatosis) disease processes associated with B. quintana infection. The acute disease (trench fever) may be characterized by fever, headache, and leg pain (shin bone disease) subsequent to the bite of an infected human body louse (Pediculus humanus humanus).

### Cat Scratch Disease (CSD)

In some embodiments, the therapeutic agents of the disclosure may be used to treat Cat Scratch disease which may be caused by B. henselae.

Atypical manifestations of CSD include tonsillitis, encephalitis, cerebral arteritis, transverse myelitis, granulomatous hepatitis and/or splenitis, osteolysis, pneumonia, pleural effusion, and thrombocytopenia purpura. Therapeutic agents of the disclosure may also be used to treat a subset of patients who lack the classical fever and lymphadenopathy associated with typical CSD.

### Bacillary Angiomatosis and Endocarditis

In some embodiments, the therapeutic agents of the disclosure may be used to treat Bacillary angiomatosis (caused by B. henselae or B. quintana) and/or bacillary peliosis (caused by B. henselae) which occur primarily in people with weakened immune systems, such as those with advanced HIV infection. Bacillary angiomatosis may result in lesions in the skin, under the skin, in bone, or in other organs.

In some embodiments, the therapeutic agents of the disclosure may be used to treat endocarditis associated with Bartonella infection.

### Formulations

In some embodiments, therapeutic agents are administered to humans, human patients or subjects. For the purposes of the present disclosure, the phrase “active ingredient” generally refers to the therapeutic agents to be delivered as described herein.

Although the descriptions of formulations provided herein are principally directed to formulations which are suitable for administration to humans, it will be understood by the skilled artisan that such therapeutic agents are generally suitable for administration to any other animal, e.g., to non-human animals, e.g., non-human mammals. Modification of formulations suitable for administration to humans in order to render the therapeutic agents suitable for administration to various animals is well understood, and the ordinarily skilled veterinary pharmacologist can design and/or perform such modification with merely ordinary, if any, experimentation. Subjects to which administration of the formulations is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the therapeutic agents described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A formulation in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a formulation in accordance with the disclosure will vary, depending upon the identity, size, and/or condition of the subject treated and further depending upon the route by which the formulation is to be administered. By way of example, the formulation may include between about 0.1% and about 100%, e.g., between about 0.5% and about 50%, between about 1% and about 30%, between about 5% and about 80%, or, in some embodiments, at least 20%, at least 40%, at least 60%, or at least 80% (w/w) active ingredient.

The therapeutic agents of the present disclosure can be formulated using one or more excipients to: (1) increase stability; (2) permit the sustained or delayed release; (3) alter the biodistribution; (4) alter the release profile of the therapeutic agents in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present disclosure may also include, without limitation, lipidoids, liposomes, lipid nanoparticles, polymers, lipoplexes, core-shell nanoparticles, peptides, proteins, hyaluronidase, nanoparticle mimics and combinations thereof.

In some embodiments, pharmaceutical compositions or formulations of the disclosure may be adapted to deliver a prescribed dosage of one or more therapeutic agents to a cell, a group of cells, an organ or tissue, an animal or a human. Methods of incorporating therapeutic agents into pharmaceutical preparations are widely known in the art. The pharmaceutical formulation may include excipients, such as without limitation, binders, coating, disintegrants, fillers, diluents, flavors, colors, lubricants, glidants, preservatives, sorbents, sweeteners, conjugated linoleic acid (CLA), gelatin, beeswax, purified water, glycerol, any type of oil, including, without limitation, fish oil or soybean oil, or the like.

Therapeutic agents and/or pharmaceutical formulations can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols. It will further be appreciated by an ordinary practitioner of the art that the term also encompasses those therapeutic agents and/or pharmaceutical formulations that contain an admixture of two or more pharmacologically active compounds, such compounds being administered, for example, as a combination therapy.

A pharmaceutical formulation in accordance with the present disclosure may be prepared, packaged, and/or sold in bulk, as a single unit dose, and/or as a plurality of single unit doses. As used herein, a “unit dose” refers to a discrete amount of the pharmaceutical formulation comprising a predetermined amount of therapeutic agent or other compounds. The amount of therapeutic agent may generally be equal to the dosage of therapeutic agent administered to a subject and/or a convenient fraction of such dosage including, but not limited to, one-half or one-third of such a dosage.

### Excipients

Formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference in its entirety) discloses various excipients used in formulating pharmaceutical compositions and techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof. Excipients such as cocoa butter and suppository waxes, coloring agents, coating agents, sweetening, flavoring, and/or perfuming agents can be present in the composition, according to the judgment of the formulator.

### Administration

In some embodiments, therapeutic agents and/or pharmaceutical formulations that include therapeutic agents may be administered according to one or more administration routes. In some embodiments, administration is enteral (into the intestine), transdermal, intravenous bolus, intralesional (within or introduced directly to a localized lesion), intrapulmonary (within the lungs or its bronchi), diagnostic, intraocular (within the eye), transtympanic (across or through the tympanic cavity), intravesical infusion, sublingual, nasogastric (through the nose and into the stomach), spinal, intracartilaginous (within a cartilage), insufflation (snorting), rectal, intravascular (within a vessel or vessels), buccal (directed toward the check), dental (to a tooth or teeth), intratesticular (within the testicle), intratympanic (within the aurus media), percutaneous, intrathoracic (within the thorax), submucosal, cutaneous, epicutaneous (application onto the skin), dental intracornal, intramedullary (within the marrow cavity of a bone), intra-abdominal, epidural (into the dura matter), intramuscular (into a muscle), intralymphatic (within the lymph), iontophoresis (by means of electric current where ions of soluble salts migrate into the tissues of the body), subcutaneous (under the skin), intragastric (within the stomach), nasal administration (through the nose), transvaginal, intravenous drip, endosinusial, intraprostatic (within the prostate gland), soft tissue, intradural (within or beneath the dura), subconjunctival, oral (by way of the mouth), peridural, parenteral, intraduodenal (within the duodenum), intracisternal (within the cisterna magna cerebellomedularis), periodontal, periarticular, biliary perfusion, intracoronary (within the coronary arteries), intrathecal (within the cerebrospinal fluid at any level of the cerebrospinal axis), intrameningeal (within the meninges), intracavernous injection (into a pathologic cavity) intracavitary (into the base of the penis), intrabiliary, subarachnoid, intrabursal, ureteral (to the ureter), intratendinous (within a tendon), auricular (in or by way of the car), intracardiac (into the heart), enema, intraepidermal (to the epidermis), intraventricular (within a ventricle), intramyocardial (within the myocardium), intratubular (within the tubules of an organ), vaginal, sublabial, intracorporus cavernosum (within the dilatable spaces of the corporus cavernosa of the penis), intradermal (into the skin itself), intravitreal (through the eye), perineural, cardiac perfusion, irrigation (to bathe or flush open wounds or body cavities), in car drops, endotracheal, intraosseous infusion (into the bone marrow), caudal block, intrauterine, transtracheal (through the wall of the trachea), intra-articular, intracorneal (within the cornea), endocervical, extracorporeal, intraspinal (within the vertebral column), transmucosal (diffusion through a mucous membrane), topical, photopheresis, oropharyngeal (directly to the mouth and pharynx), occlusive dressing technique (topical route administration which is then covered by a dressing which occludes the area), transplacental (through or across the placenta), intrapericardial (within the pericardium), intraarterial (into an artery), interstitial, intracerebral (into the cerebrum), intracerebroventricular (into the cerebral ventricles), intrapleural (within the pleura), infiltration, intrabronchial, intrasinal (within the nasal or periorbital sinuses), intraductal (within a duct of a gland), intracaudal (within the cauda equine), nerve block, retrobulbar (behind the pons or behind the eyeball), intravenous (into a vein), intra-amniotic, conjunctival, intrasynovial (within the synovial cavity of a joint), gastroenteral, intraluminal (within a lumen of a tube), electro-osmosis, intrailcal (within the distal portion of the small intestine), intraesophageal (to the esophagus), extra-amniotic administration, hemodialysis, intragingival (within the gingivae), intratumor (within a tumor), eye drops (onto the conjunctiva), laryngeal (directly upon the larynx), urethral (to the urethra), intravaginal administration, intraperitoneal (infusion or injection into the peritoneum), respiratory (within the respiratory tract by inhaling orally or nasally for local or systemic effect), intradiscal (within a disc), ophthalmic (to the external eye), and/or intraovarian (within the ovary).

In some embodiments, therapeutic agents and/or pharmaceutical formulations that include therapeutic agents may be administered by intraarticular administration, extracorporeal administration, intrabronchial administration, endocervical administration, endosinusial administration, endotracheal administration, enteral administration, epidural administration, intra-abdominal administration, intrabiliary administration, intrabursal administration, oropharyngeal administration, interstitial administration, intracardiac administration, intracartilaginous administration, intracaudal administration, intracavernous administration, intracerebral administration, intracorporous cavernosum, intracavitary administration, intracorneal administration, intracisternal administration, cranial administration, intracranial administration, intradermal administration, intralesional administration, intratympanic administration, intragingival administration, intraocular administration, intradiscal administration, intraductal administration, intraduodenal administration, ophthalmic administration, intradural administration, intraepidermal administration, intraesophageal administration, nasogastric administration, nasal administration, laryngeal administration, intraventricular administration, intragastric administration, intrahepatic administration, intraluminal administration, intravitreal administration, intravesicular administration, intralymphatic administration, intramammary administration, intramedullary administration, intrasinal administration, intrameningcal administration, intranodal administration, intraovarian administration, intraperitoneal administration, intrapleural administration, intraprostatic administration, intraluminal administration, intraspinal administration, intrasynovial administration, intratendinous administration, intratesticular administration, subconjunctival administration, intracerebroventricular administration, epicutancous administration, intravenous administration, retrobulbar administration, periarticular administration, intrathoracic administration, subarachnoid administration, intratubular administration, periodontal administration, transtympanic administration, transtracheal administration, intratumor administration, vaginal administration, urethral administration, intrauterine administration, oral administration, gastroenteral administration, parenteral administration, sublingual administration, ureteral administration, percutaneous administration, peridural administration, transmucosal administration, perineural administration, transdermal administration, rectal administration, soft tissue administration, intraarterial administration, subcutaneous administration, topical administration, extra-amniotic administration, car drops, or intravesical infusion.

Therapeutic agents and/or pharmaceutical formulations of the present disclosure may be administered orally but any suitable route of administration may be employed for providing a subject with an effective dosage of drugs of the chemical compositions described herein. For example, oral, rectal, topical, parenteral, ocular, pulmonary, nasal, and the like may be employed. Dosage forms include tablets, troches, dispersions, suspensions, solutions, capsules, creams, ointments, acrosols, and the like. In certain embodiments, it may be advantageous that the compositions described herein be administered orally.

Therapeutic agents and/or pharmaceutical formulations of the present disclosure may be administered in the conventional manner by any route where they are active. Administration can be systemic, parenteral, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration of the composition of the present disclosure (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

For administration by inhalation or intranasal, the pharmaceutical formulation may be delivered in the form of an aerosol spray presentation from pressurized packs or nebulizers. The compounds may also be delivered in the form of a cream, liquid, spray, powder, or suppository. A metered dose of the formulation can be provided from a reservoir of the formulation. In addition, predetermined dosages can be provided, for example, suppository forms can be provided for insertion into the nose having a predetermined dosage. Kits can be provided, where prepared dosage forms and instructions for administering the dosages are included.

Suitable topical formulations for use in the present embodiments may also include transdermal devices, acrosols, creams, ointments, lotions, dusting powders, gels, and the like.

### Dosing

Therapeutic agents and/or pharmaceutical formulations described herein may be administered to a subject using any amount and any route of administration effective for treating a disease, disorder, and/or condition. The exact amount required will vary from subject to subject, depending on the species, age, and general condition of the subject, the severity of the disease, the particular formulation, its mode of administration, its mode of activity, and the like.

In some embodiments, formulations in accordance with the present disclosure may be administered at dosage levels sufficient to deliver a therapeutic agent dose of about 0.1 mg/kg to about 500 mg/kg body weight, from about 0.1 mg/kg to about 250 mg/kg body weight, from about 0.1 mg/kg to about 100 mg/kg body weight, from about 0.1 mg/kg to about 50 mg/kg body weight, from about 0.1 mg/kg to about 10 mg/kg body weight, and/or about 0.1 mg/kg to about 5 mg/kg body weight, from about 1 mg/kg to about 2 mg/kg body weight, from about 1 mg/kg to about 10 mg/kg, from about 5mg/kg to about 15mg//kg, from about 10 mg/kg to about 20 mg/kg body weight, from about 20 mg/kg to about 30 mg/kg body weight, from about 30 mg/kg to about 40 mg/kg body weight, from about 40 mg/kg to about 50 mg/kg body weight, from about 50 mg/kg to about 60 mg/kg body weight, from about 60 mg/kg to about 70 mg/kg body weight, from about 70 mg/kg to about 80 mg/kg body weight, from about 80 mg/kg to about 90 mg/kg body weight, from about 90 mg/kg to about 100 mg/kg body weight, from about 100 mg/kg to about 110 mg/kg body weight, from about 110 mg/kg to about 120 mg/kg body weight, from about 120 mg/kg to about 130 mg/kg body weight, from about 130 mg/kg to about 140 mg/kg body weight, from about 140 mg/kg to about 150 mg/kg body weight, from about 150 mg/kg to about 160 mg/kg body weight, from about 160 mg/kg to about 170 mg/kg body weight, from about 170 mg/kg to about 180 mg/kg body weight, from about 180 mg/kg to about 190 mg/kg body weight, from about 190 mg/kg to about 200 mg/kg body weight, from about 15 mg/kg to about 25 mg/kg body weight, from about 25 mg/kg to about 35 mg/kg body weight, from about 35 mg/kg to about 45 mg/kg body weight, from about 45 mg/kg to about 55 mg/kg body weight, from about 55 mg/kg to about 65 mg/kg body weight, from about 65 mg/kg to about 75 mg/kg body weight, from about 75 mg/kg to about 85 mg/kg body weight, from about 85 mg/kg to about 95 mg/kg body weight, from about 95 mg/kg to about 105 mg/kg body weight, from about 105 mg/kg to about 115 mg/kg body weight, from about 115 mg/kg to about 125 mg/kg body weight, from about 125 mg/kg to about 135 mg/kg body weight, from about 135 mg/kg to about 145 mg/kg body weight, from about 145 mg/kg to about 155 mg/kg body weight, from about 155 mg/kg to about 165 mg/kg body weight, from about 165 mg/kg to about 175 mg/kg body weight, from about 175 mg/kg to about 185 mg/kg body weight, from about 185 mg/kg to about 195 mg/kg body weight, from about 195 mg/kg to about 205 mg/kg body weight.

In some embodiments, therapeutic agents described herein may be administered at a dose of about 10-50 μg/mL, 20 μg/mL, or 40 μg/mL.

In some embodiments, therapeutic agents and/or pharmaceutical formulations of the present disclosure are provided in one or more doses and are administered one or more times to subjects. Some therapeutic agents and/or pharmaceutical formulations are provided in only a single administration. Some therapeutic agents and/or pharmaceutical formulations are provided according to a dosing schedule that include two or more administrations. Each administration may be at the same dose or may be different from a previous and/or subsequent dose. In some embodiments, subjects are provided an initial dose that is higher than subsequent doses (referred to herein as a “loading dose”). In some embodiments, doses are decreased over the course of administration. In some embodiments, dosing schedules include pharmaceutical formulation administration from about every 2 hours to about every 10 hours, from about every 4 hours to about every 20 hours, from about every 6 hours to about every 30 hours, from about every 8 hours to about every 40 hours, from about every 10 hours to about every 50 hours, from about every 12 hours to about every 60 hours, from about every 14 hours to about every 70 hours, from about every 16 hours to about every 80 hours, from about every 18 hours to about every 90 hours, from about every 20 hours to about every 100 hours, from about every 22 hours to about every 120 hours, from about every 24 hours to about every 132 hours, from about every 30 hours to about every 144 hours, from about every 36 hours to about every 156 hours, from about every 48 hours to about every 168 hours, from about every 2 days to about every 10 days, from about every 4 days to about every 15 days, from about every 6 days to about every 20 days, from about every 8 days to about every 25 days, from about every 10 days to about every 30 days, from about every 12 days to about every 35 days, from about every 14 days to about every 40 days, from about every 16 days to about every 45 days, from about every 18 days to about every 50 days, from about every 20 days to about every 55 days, from about every 22 days to about every 60 days, from about every 24 days to about every 65 days, from about every 30 days to about every 70 days, from about every 2 weeks to about every 8 weeks, from about every 3 weeks to about every 12 weeks, from about every 4 weeks to about every 16 weeks, from about every 5 weeks to about every 20 weeks, from about every 6 weeks to about every 24 weeks, from about every 7 weeks to about every 28 weeks, from about every 8 weeks to about every 32 weeks, from about every 9 weeks to about every 36 weeks, from about every 10 weeks to about every 40 weeks, from about every 11 weeks to about every 44 weeks, from about every 12 weeks to about every 48 weeks, from about every 14 weeks to about every 52 weeks, from about every 16 weeks to about every 56 weeks, from about every 20 weeks to about every 60 weeks, from about every 2 months to about every 6 months, from about every 3 months to about every 12 months, from about every 4 months to about every 18 months, from about every 5 months to about every 24 months, from about every 6 months to about every 30 months, from about every 7 months to about every 36 months, from about every 8 months to about every 42 months, from about every 9 months to about every 48 months, from about every 10 months to about every 54 months, from about every 11 months to about every 60 months, from about every 12 months to about every 66 months, from about 2 years to about 5 years, from about 3 years to about 10 years, from about 4 years to about 15 years, from about 5 years to about 20 years, from about 6 years to about 25 years, from about 7 years to about 30 years, from about 8 years to about 35 years, from about 9 years to about 40 years, from about 10 years to about 45 years, from about 15 years to about 50 years, or more than every 50 years.

The desired dosage may be delivered for a duration of about 5 days to 365 days, about 5 days to 300 days, about 5 days to 300 days, about 5 days to 250 days, about 5 days to 200 days, about 5 days to 100 days, about 5 days to 60 days, about days to 30 days, about 5 days to 14 days, or about 3 days to 7 days, preferably about 21 days to 28 days.

In some embodiments, the desired dosage of the formulations described herein may be administered once daily or multiple times in a day. For example, a treatment regimen may include administering a dosage level sufficient to deliver 10 mg/kg body weight twice daily, 20 mg/kg body weight twice daily, 50 mg/kg body weight once daily, 10 mg/kg body weight three times daily, 20 mg/kg body weight four times daily, or 50 mg/kg body weight twice daily.

### Pulse Dosing

In some embodiments, subjects may be administered a pulse dose of the therapeutic agents and/or pharmaceutical formulations of the present disclosure. As used herein, “pulse” refers to the plurality of doses at spaced apart time intervals. Generally, upon administration of the first dose, the growth of Bartonella may be inhibited, retarded and/or the bacteria may be killed. Following, the first dose, the Bartonella bacteria levels may increase; and a second dose may be initiated. Eradication of Bartonella may therefore be achieved by several rounds of pulse dosing. Pulse dosing may be employed to eliminate persistent Bartonella. In some embodiments, pulse dosing schedules include pharmaceutical formulation administration from about every 2 hours to about every 10 hours, from about every 4 hours to about every 20 hours, from about every 6 hours to about every 30 hours, from about every 8 hours to about every 40 hours, from about every 10 hours to about every 50 hours, from about every 12 hours to about every 60 hours, from about every 14 hours to about every 70 hours, from about every 16 hours to about every 80 hours, from about every 18 hours to about every 90 hours, from about every 20 hours to about every 100 hours, from about every 22 hours to about every 120 hours, from about every 24 hours to about every 132 hours, from about every 30 hours to about every 144 hours, from about every 36 hours to about every 156 hours, from about every 48 hours to about every 168 hours, from about every 2 days to about every 10 days, from about every 4 days to about every 15 days, from about every 6 days to about every 20 days, from about every 8 days to about every 25 days, from about every 10 days to about every 30 days, from about every 12 days to about every 35 days, from about every 14 days to about every 40 days, from about every 16 days to about every 45 days, from about every 18 days to about every 50 days, from about every 20 days to about every 55 days, from about every 22 days to about every 60 days, from about every 24 days to about every 65 days, from about every 30 days to about every 70 days, from about every 2 weeks to about every 8 weeks, from about every 3 weeks to about every 12 weeks, from about every 4 weeks to about every 16 weeks, from about every 5 weeks to about every 20 weeks, from about every 6 weeks to about every 24 weeks, from about every 7 weeks to about every 28 weeks, from about every 8 weeks to about every 32 weeks, from about every 9 weeks to about every 36 weeks, from about every 10 weeks to about every 40 weeks, from about every 11 weeks to about every 44 weeks, from about every 12 weeks to about every 48 weeks, from about every 14 weeks to about every 52 weeks, from about every 16 weeks to about every 56 weeks, from about every 20 weeks to about every 60 weeks, from about every 2 months to about every 6 months, from about every 3 months to about every 12 months, from about every 4 months to about every 18 months, from about every 5 months to about every 24 months, from about every 6 months to about every 30 months, from about every 7 months to about every 36 months, from about every 8 months to about every 42 months, from about every 9 months to about every 48 months, from about every 10 months to about every 54 months, from about every 11 months to about every 60 months, from about every 12 months to about every 66 months, from about 2 years to about 5 years, from about 3 years to about 10 years, from about 4 years to about 15 years, from about 5 years to about 20 years, from about 6 years to about 25 years, from about 7 years to about 30 years, from about 8 years to about 35 years, from about 9 years to about 40 years, from about 10 years to about 45 years, from about 15 years to about 50 years, or more than every 50 years.

### Combinations

In some embodiments, the therapeutic agents and/or pharmaceutical formulations of the present disclosure may be used in combination with additional active agents such as antibiotics and/or vaccines. By “in combination with,” it is not intended to imply that the agents must be administered at the same time and/or formulated for delivery together, although these methods of delivery are within the scope of the present disclosure.

In some embodiments, the present disclosure encompasses the delivery of pharmaceutical, prophylactic, research, or diagnostic formulations in combination with agents that may improve their bioavailability, reduce and/or modify their metabolism, inhibit their excretion, and/or modify their distribution within the body.

The formulations of the present disclosure and the additional active agents may be administered simultaneously, sequentially, or in any order. The formulations of the present disclosure and additional active agents may be administered at different dosages, with different dosing frequencies and/or different routes, whichever is suitable. The term “administered simultaneously,” as used herein, may mean that formulations of the present disclosure and the additional active agent may be substantially administered at the same time, e.g., as a mixture or in immediate subsequent sequence. The term “administered sequentially,” as used herein, may mean that the formulations of the present disclosure and the additional active agent may not be administered at the same time but one after the other, or in groups, with a specific time interval between administrations. The time interval may be the same or different between the respective administrations of the formulations of the present disclosure and the additional active agent and may be selected, for example, from the range of 2 minutes to 96 hours, 1 to 7 days, or one, two or three weeks. Generally, the time interval between the administrations may be in the range of a few minutes to hours, such as in the range of about 2 minutes to about 72 hours, about 30 minutes to about 24 hours, or about 1 hour to about 12 hours.

Combinations of therapeutic agents and methods of treatment by administering the combinations of therapeutic agents including one or more of azlocillin, azithromycin, ceftriaxone, doxycycline, gentamicin and ampicillin are provided herein. For instance, azlocillin may be administered to a subject in combination with azithromycin. In some embodiments, azlocillin may be administered to a subject in combination with ceftriaxone. In some embodiments, azlocillin may be administered to a subject in combination with doxycycline. In some embodiments, azlocillin may be administered to a subject in combination with gentamicin. In some embodiments, azlocillin may be administered to a subject in combination with ampicillin. In some embodiments, azlocillin may be administered to a subject in combination with two or more of azithromycin, ceftriaxone, doxycycline, gentamicin and ampicillin.

### Definitions

The terms “administer,” “administration,” “administering,” and the like, when used in conjunction with a therapeutic agent means to deliver a therapeutic agent to a subject whereby the therapeutic agent positively impacts, i.e., has a therapeutic effect on, the subject or the tissue or the organ to which it is targeted. The therapeutic agents described herein can be administered either alone or in combination (concurrently or serially) and/or with other pharmaceuticals. For example, the therapeutic agents can be administered in combination with vaccines, antibiotics, antiviral agents, anti-cancer or anti-neoplastic agents, or in combination with other treatment modalities such as herbal therapy, acupuncture, naturopathy, etc.

The term “effective amount” as used herein generally refers to an amount of the therapeutic agent that is administered to decrease, prevent or inhibit the disease. The amount will vary for each compound and upon known factors related to the item or use to which the therapeutic agent is applied.

The term “immune response” as used herein refers to activity of the cells of the immune system upon exposure to a stimulus such as but not limited to an antigen. In some embodiments, the antigen may be derived from Bartonella spp.

The term “modulation” refers to up regulation (i.e., activation or stimulation), down regulation (i.e., inhibition or suppression) of a response, or the two in combination or apart.

The term “pharmaceutically acceptable” as used herein, refers to compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration. A “pharmaceutically acceptable carrier” as used herein, refers to all components of a pharmaceutical formulation that facilitate the delivery of the composition in vivo. Pharmaceutically acceptable carriers include, but are not limited to, diluents, preservatives, binders, lubricants, disintegrators, swelling agents, fillers, stabilizers, and combinations thereof.

The term “prodrug” refers to an agent, including a compound, nucleic acid, or protein that is converted into a biologically active form in vitro and/or in vivo. Prodrugs can be useful because, in some situations, they may be easier to administer than the parent compound. For example, a prodrug may be bioavailable by oral administration whereas the parent compound is not. The prodrug may also have improved solubility in pharmaceutical compositions compared to the parent drug. A prodrug may be converted into the parent drug by various mechanisms, including enzymatic processes and metabolic hydrolysis. Harper, N.J. (1962) Drug Latentiation in Jucker, ed. Progress in Drug Research, 4:221-294; Morozowich et al. (1977) Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977) Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997) Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of B-Lactam antibiotics, Pharm. Biotech. 11:345-365; Gaignault et al. (1996) Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lec and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogs, Adv. Drug Delivery Rev., 39(1-3): 183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996) Improved oral drug delivery: solubility limitations overcome by the use of prodrugs, Adv. Drug Delivery Rev. 19(2): 115-130; Fleisher et al. (1985) Design of prodrugs for improved gastrointestinal absorption by intestinal enzyme targeting, Methods Enzymol. 112: 360-81; Farquhar D, et al. (1983) Biologically Reversible Phosphate-Protective Groups, J. Pharm. Sci., 72(3): 324-325; Han, H. K. et al. (2000) Targeted prodrug design to optimize drug delivery, AAPS PharmSci., 2(1): E6; Sadzuka Y. (2000) Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl. 2: S15-27; Wang, W. et al. (1999) Prodrug approaches to the improved delivery of peptide drugs. Curr. Pharm. Des., 5(4):265-87.

A “subject” may include a human subject for medical purposes, such as for the treatment of an existing disease, disorder, condition or the prophylactic for preventing the onset of a disease, disorder, or condition or an animal subject for medical, veterinary purposes, or developmental purposes. Suitable animal subjects include mammals, including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

As used herein, the term “therapeutic agent” refers to any substance used to restore or promote the health and/or wellbeing of a subject and/or to treat, prevent, alleviate, cure, or diagnose a disease, disorder, or condition.

The terms “treatment,” “treating,” and the like, refer to an intervention performed with the intention of preventing the development or altering the pathology or symptoms of a disorder. Accordingly, “treatment” can refer to therapeutic treatment or prophylactic or preventative measures. In some embodiments, the treatment is for therapeutic treatment. In some embodiments, the treatment is for prophylactic or preventative treatment. Those in need of treatment can include those already with the disorder as well as those in which the disorder is to be prevented. In some embodiments, the treatment is for experimental treatment.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

### EXAMPLES

### Example 1. Experimental Methods

Provided herein are experimental methods utilized to obtain the results described in Example 2, Example 3, Example 4 and Example 5.

### Bacterial Strains and Growth Conditions

The B. henselae strains used were San Antonio 2, 267HO04, a human clinical isolate and B. vinsonii subsp. berkhoffii. Both the bacteria were initially grown on tryptic soy agar (TSA) supplemented with 5% defibrinated sheep blood plates that were either pre-made (Remel, R01198) or homemade, in a humidified atmosphere at 37° C. with 5% CO₂ for 7 days. For the preparation of liquid cultures, an individual well-grown colony from the plate was resuspended in 15 ml Falcon tubes containing 3 mL of either Schneider's insect media supplemented with 10% FBS, or Grace's insect media (Gibco, 11605-094) supplemented with 10% FBS, or BAPGM serum-free liquid media. The cultures grew for 7 more days in the atmosphere as above without shaking. The number of viable bacteria was determined by either plating serial dilutions of the culture on plates for CFU or by live/dead staining. Before every experiment, bacteria were mixed well and passed through 22-gauge needle to break large clumps of bacteria.

### Live/Dead Staining

To determine the viability of the bacteria grown in liquid culture media, LIVE/DEAD BacLight™ Bacterial viability kit for microscopy (ThermoFisher, L7012) was used according to the manufacturer's protocol. Briefly, 7-day old liquid culture media was used for the determination of the viability. Initially, bacteria were diluted down to an optical density (OD) of 0.1 using the respective liquid culture media. 1 ml of 0.1 OD bacterial culture was then transferred into a 2 ml microcentrifuge tube and centrifuged at 7000 rpm for 10 min. After removing the supernatant, the pellet was resuspended in 1 ml of 0.85% NaCl and bacteria was pelleted again. After three washes, bacteria were resuspended in 1 ml of 0.85% NaCl and incubated with 1 μl of green-fluorescent nucleic acid dye (SYTO9) and 1 μl of red-fluorescent nucleic acid dye (PI) for 15 minutes. All the incubations were performed at room temperature and in the dark. After 15 minutes of incubation with both the dyes, bacteria were visualized under fluorescence microscopy and images were acquired using Nikon NIS elements software. Later, mean fluorescence intensity of both the SYTO9 and PI was measured using the Nikon elements software. Mean fluorescence values were acquired from a total of 10 images per experimental condition. To obtain dead cell counts, bacteria were treated with 70% isopropanol and incubated for 1 hr, while the sample was mixed for every 15 min and stained as above. Additionally, the entire staining protocol was repeated by staining the bacteria with PI first and then with SYTO9 both on dead and live bacteria and the fluorescence values were recorded accordingly. However, in the staining of therapeutic agent treated bacteria, both the dyes were pre-mixed into 1 ml of sterile-filtered distilled water, as previously described and 10 μl of the mixture was added into each well, incubated and visualized as described above.

### H82 Cell Culture and Seeding Density

DH82 are a canine cell line having a macrophage-like morphology. Cells were initially cultured in T75-Flasks (Corning, 430641U) until confluency was reached and were split at a ratio of 1:4 for every 2-3 days. Cells were then seeded into either 24-well cell culture plates (Corning, 3526) for coculture assay or 8-well chamber slides (Millipore #PEGGS0816) for immunofluorescence assays. A total of 50,000 cells were seeded into each well of a 24-well plate or per chamber of an 8-well chamber slide with EMEM with 15% FBS and incubated for 24-48 hours before infections.

### Minimum Inhibitory Concentration (MIC) and Minimum Bactericidal Concentration (MBC) Assays

Optical density (OD) of 7-day old bacterial liquid culture that was grown from an individual colony was measured using a spectrophotometer (Bio-Rad, SmartSpec 300, 170-2501). Bacterial culture was then diluted down to an OD of 0.1 [symbol] 3×10⁸ cfu/ml with the respective media.

To determine the MIC of the antibiotics and for the visualization of the bacterial growth, a clear 96-well plate was used. Into each well of a 96-well plate, 50 μl of the 0.1 OD bacterial culture and 50 μl of the liquid media containing antibiotics were added to the wells bringing the total volume to 100 μl of 0.05 OD bacteria (CFU [symbol] 1.5×10⁷) in each well. Wells containing only bacteria with solvent used in the dilution of the antibiotics served as a positive control to verify the growth of bacteria. As a negative control, wells containing only media and solvents were used. Each concentration of diluted antibiotic was run in 4 replicates to minimize the error rate. The 96-well plate was covered with the lid and incubated at 37° C. with 5% CO₂. OD of the culture was read after 48 hrs and 96 hrs using a hybrid multi-mode microplate reader (Biotek, Synergy H4 hybrid reader) at OD₆₀₀. The assay was repeated three times.

For the MBC assay, the therapeutic agent concentrations that showed inhibition of bacterial growth with MIC assay were used. Briefly, the bacterial culture that was treated with therapeutic agents for 96 hrs was collected into a 1.5 ml microcentrifuge tubes and centrifuged at 7000 rpm for 10 min. The supernatant was removed, and the bacterial pellet was washed with 1×PBS twice. The bacterial pellet was then resuspended in 100 μL of 1×PBS, plated onto blood agar plates and incubated at 37° C. with 5% CO₂ for about 14 days. This assay was repeated three times with all the 6 different antibiotics (azlocillin, azithromycin, ceftriaxone, doxycycline, gentamicin and ampicillin) and at different concentrations.

### Therapeutic Agents and Their Dilution

Therapeutic agent stocks were prepared in the following solvents: 10 mg/ml of ceftriaxone (USP), doxycycline (Fisher scientific, D9891-1G), gentamicin (Fisher scientific, 1405-41-0), ampicillin (Fisher scientific, 69-52-3) and azlocillin (Flightpath) in sterile cell culture grade water, whereas 5 mg/ml of azithromycin (Sigma, PZ0007-5MG) was dissolved in sterile cell culture grade DMSO. Antibiotics were then aliquoted and stored at either −80° C. or −20° C. until further use. On the day of assay, these stock concentrations were diluted to 1 mg/ml and 100 μg/ml in the respected bacterial culture media for MIC and cell-free, liquid culture MBC assays. For the DH82 cell-based MBC assay, antibiotics were diluted in EMEM media supplemented with 15% heat inactivated FBS.

### DH82 Cell-Based MBC Assay

Bacteria (B. henselae) that were grown in Grace's insect media, were centrifuged at 7000 rpm for 10 min. The supernatant was removed and washed with 1×PBS for three times. After the washes, pellet was resuspended in EMEM media supplemented with 15% heat inactivated FBS. This bacterial suspension was then added to DH82 cells at a multiplicity of infection (MOI) of 1:50 and incubated for 2 hours. After 2 hours, unbound bacteria were removed by washing the cells with only EMEM media 3 times each 15 min wash. Cells were then incubated with EMEM+15% FBS media at 37° C. with 5% CO₂ for 48 hours.

After 48 hrs of incubation, the media was removed from the cells and the therapeutic agents that were diluted in EMEM+15% FBS was added. Each therapeutic agent was diluted at 11 different concentrations (16, 8, 4, 2, 1, 0.5, 0.25, 0.125, 0.062, 0.031, 0.0156 μg/ml), as described above and incubated for 96 hours. This incubation period was determined based on cell-free, liquid culture MBC data. The bactericidal activity of the therapeutic agents were then evaluated by plating the lysed cells on blood agar plates. Briefly, infected DH82 cells were initially washed for 30 min twice to remove the antibiotics that were added. Later, cells were osmotically lysed by adding 1 ml of sterile ice-cold distilled water and incubated for 5 min on ice. The lysed cells were then plated onto blood agar plates and incubated at 37° C. with 5% CO₂ for about 10 days to evaluate the growth of viable B. henselae. This experiment was conducted in duplicates and repeated twice with all 6 antibiotics including 11 different concentrations. The untreated infected cells were used as a positive control and untreated uninfected cells served as a negative control. The CFU counts are presented as average±standard deviation.

### Immunofluorescence Assay

After 48 hours of infection with B. henselae, DH82 cells were prepared for immunofluorescence assay. The seeding of the cells and the infection was performed on 8-well chamber slides (Millipore #PEGGS0816). Initially, cells were fixed with chilled ethanol and acetic acid at a ratio of 2:1 and incubated at −20° C. for 10 mins. Cells were washed with 1×PBS three times by gentle rocking. Next, cells were permeabilized using 0.1% Triton-X 100 dissolved in 1×PBS and incubated for 20 min. After 20 min, Triton-X was washed from the cells using 1×PBS. Cells were then covered with blocking buffer (10% Normal Goat Serum-Gibco, 16-210-072, in PBS) for one hour at room temperature. After blocking, cells were incubated with the primary antibody: rabbit polyclonal anti-B. henselae (serum derived from a hyperimmunized rabbit 8 weeks after inoculation with in vitro propagated B. henselae) at a dilution of 1:300 for 1 hour followed by three 1×PBS washes. Cells were then incubated with secondary antibody Goat Anti-rabbit IgG-Alexa Fluor 594 (Thermo Fisher Scientific, R37117) diluted 1:1000 in blocking buffer. Slides were washed similar to after incubation with primary antibody, then partially dried by tapping the glass edge onto a paper towel. Cells were counterstained with DAPI for 10 min to stain nuclei (EMD Millipore, 2160) and mounted with anti-quenching solution (ThermoFisher, P36934) and placed under a coverslip. To prevent the loss of fluorescent signal intensity, slides were examined and photographed within one week of completion of the staining procedure. As a negative control, uninfected cells were used. The experiment was repeated three times and the percentage of infection was reported as average±standard deviation.

Imaging was performed using a Nikon Ti2-E motorized fluorescence microscopy. Infected and uninfected cells were imaged during the same session with identical acquisition parameters. Fluorescence intensity was optimized using uninfected cells to account for autofluorescence from the cells and was found to remain constant for all the infected cells. Any adjustments to brightness, contrast, or color balance were applied to the whole image.

### Example 2: Determination of Minimum Inhibitory Concentration (MIC)

The determination of susceptibility to therapeutic agents was performed in liquid culture media. Six different therapeutic agents were tested with multiple different concentrations against B. henselae and B. vinsonii. Therapeutic agent concentrations tested were as follows: Ceftriaxone 0.001, 0.01, 0.1, 0.3, 0.5, 1, 2, and 5 μg/ml; doxycycline 0.001, 0.01, 0.1, 0.3, 0.5, 1, 2, 5, and 10 μg/ml, gentamicin: 0.01, 0.1, 0.3, 0.5, 1, 2, 4, 8, and 16 μg/ml, and azithromycin: 0.001, 0.005, 0.02, 0.1, 0.3, 0.5, 1, 2, 5, and 10 μg/ml, were tested based on previous antibiotic susceptibility studies. The following azlocillin dilutions were included: 0.001, 0.005, 0.01, 0.02, 0.5, 1, 2, and 5 μg/ml. The ampicillin dilutions included were as follows: 0.001, 0.005, 0.01, 0.02, 0.5, 1, 2, 4, and 8 μg/ml. Both the species of Bartonella were susceptible to all the antibiotics that were used in BAPGM and Grace's culture liquid mediums. MICs ranged from 0.01 to 0.1 μg/ml for ceftriaxone, doxycycline, and ampicillin, 2.0-4.0 μg/ml for gentamicin, 0.1-0.5 μg/ml for azithromycin, and 0.001-0.01 μg/ml for azlocillin. Among all the antibiotics, azlocillin was most effective in inhibiting the growth of both the species of Bartonella. However, both the bacteria were not susceptible towards any of the antibiotics when tested in Schneider's insect medium (Table 1).

Table 1 shows the comparison of MICs for B. henselae and B. vinsonii in 3 different liquid culture mediums determined by measuring optical density using a hybrid multi-mode microplate reader at OD 600.

TABLE 1
MIC values

| | B. henselae | | | B. vinsonii | |
|---|---|---|---|---|---|
| Therapeutic | MIC (μg/ml) | | | | Therapeutic |
| agent | Schneider's | BAPGM | Grace's | Grace's | agent |
| Ceftriaxone | >0.3 | 0.01-0.1 | 0.01-0.1 | 0-0.01 | Ceftriaxone |
| Doxycycline | >0.3 | 0.01-0.1 | 0.01-0.1 | 0.01-0.1 | Doxycycline |
| Gentamicin | >1 | 0.1-0.5 | 2.0-4.0 | 2.0-4.0 | Gentamicin |
| Azithromycin | >0.02 | 0.005-0.02 | 0.3-0.5 | 0.1-0.3 | Azithromycin |
| Azlocillin | >2 | 0.01-0.02 | 0.005-0.01 | 0.001-0.005 | Azlocillin |
| Ampicillin | | | 0.02-0.1 | 0.01-0.02 | Ampicillin |

### Example 3: Comparison of MBC

For this assay, both B. henselae and B. vinsonii were grown in Grace's insect media and were tested against all of the 6 antibiotics (ceftriaxone, doxycycline, gentamicin, azithromycin, azlocillin, ampicillin). While both bacteria were susceptible to all of the antibiotics, doxycycline and ampicillin did not completely inhibit the growth of bacteria at concentrations of 10 μg/ml and 8 μg/ml, respectively. However, ceftriaxone, gentamicin, azithromycin, and azlocillin were all bactericidal and completely eliminated the growth at concentrations below 10 g/ml. The complete list of bactericidal concentrations of all the antibiotics is listed in Table 2. Table 2 provides comparison of MBCs for B. henselae and B. vinsonii strains in cell-free liquid culture medium determined by incubating the bacteria with antibiotics in a 96-well plate and plating on tryptic soy agar supplemented with 5% sheep blood. Additionally, MBC for B. henselae determined by DH82 cell-based assay (right column) is provided.

TABLE 2
MBC values

| | | | DH82 cell- |
|---|---|---|---|
| | Cell-free, liquid culture assay | | based assay |
| Therapeutic | MBC (μg/ml) | | MBC (μg/ml) |
| agent | B. henselae | B. vinsonii | B. henselae |
| Ceftriaxone | 2.0-4.0 | 0.5-1.0 | >16 |
| Doxycycline | >10 | >10 | >16 |
| Gentamycin | 2.0-4.0 | 4.0-8.0 | >16 |
| Azithromycin | 5.0-10 | 2.0-5.0 | >16 |
| Azlocillin | 1.0-2.0 | 0.25-0.5 | >16 |
| Ampicillin | >8 | >8 | >16 |

### Example 4: Efficacy of Therapeutic Agents in Cell-Based Co-Culture Systems

To evaluate the bactericidal activity and to determine if these antibiotics could penetrate the eukaryotic cell wall, a new DH82 cell-based coculture system was utilized. When B. henselae and DH82 cells were cocultured, an MOI of 50 was obtained, with about 83±0.091% of DH82 cells harboring intracellular B. henselae. Uninfected cells stained with primary rabbit B. henselaehyperimmunized polyclonal (primary) and goat anti-rabbit IgG Alexa fluor 594 (secondary) antibodies were used as a negative control. As an isotype control, DH82 cells infected with B. henselae and incubated with primary rabbit IgG were used. For the negative and positive control respectively, (a) DH82 cells were stained with primary rabbit B. henselae hyperimmunized polyclonal (primary) and goat anti-rabbit IgG Alexa fluor 594 (secondary) antibodies or (b) cells were stained with primary Rabbit IgG isotype control (ThermoFisher #02-6102) and secondary goat anti-rabbit IgG Alexa fluor 594 secondary antibody. Images were acquired with a Nikon fluorescence microscope using a 60×objective.

After confirming the rate of infectivity, the same MOI was used for testing therapeutic agents in this coculture system. When therapeutic agents were used individually on DH82 cell-based system, none of the therapeutic agents were potent against B. henselae at the concentration 16 μg/ml, except for azithromycin and azlocillin showed reduced bacterial growth but were unable to completely inhibit the growth. Based on these data, it was hypothesized that antibiotic combinations with azithromycin/azlocillin would effectively inhibit the growth. To determine β-lactam and macrolide combination efficacy, the combination of azithromycin and ampicillin was evaluated in parallel. The combination of azithromycin/azlocillin was able to completely eliminate the growth of B. henselae at equal concentrations of <2 μg/ml. The combination of azithromycin/ampicillin was also effective at the concentrations of 4 μg/ml. The combinations were also tested in the cell-free liquid culture medium. The combination of azithromycin/azlocillin was more effective than the azithromycin/ampicillin combination (Table 3). The azithromycin/azlocillin combination was able to eliminate the growth of B. henselae at a concentration of 1 μg/ml, whereas the azithromycin/ampicillin combination was effective at 4 μg/ml of concentration.

TABLE 3
Comparison of effect of drug combinations on B. henselae in
cell-free liquid culture- based assay and DH82 cell-based assay

| | | DH82 cell- | Cell-free, liquid |
|---|---|---|---|
| | Concentration | based assay | culture assay |
| | of each | CFU/ml after | CFU/ml after |
| | therapeutic | 96 hrs of drug | 96 hrs of drug |
| Combination | agent (μg/ml) | exposure | exposure |
| Drug free | 0 | 1.03 ± 0.76 × 10⁴ | 1 ± 0.1 × 10⁹ |
| control | | | |
| azithromycin + | 16 | 0 | 0 |
| ampicillin | 8 | 0 | 0 |
| | 4 | 0 | 3.5 ± 2.12 |
| | 2 | 10 | 37.5 ± 3.54 |
| | 1 | ND | 60.5 ± 6.36 |
| | 0.5 | ND | 1.25 ± 0.35 × 10³ |
| Drug free | 0 | 0.63 ± 0.61 × 10⁴ | 1.15 ± 0.21 × 10⁹ |
| control | | | |
| azithromycin + | 16 | 0 | 0 |
| azlocillin | 8 | 0 | 0 |
| | 4 | 0 | 0 |
| | 2 | 0 | 0 |
| | 1 | ND | 11 ± 4.24 |
| | 0.5 | ND | 2.02 ± 0.33 × 10² |

ND = not determined

### Example 5: Live/Dead Staining

Bacteria were stained with the live/dead BacLight™ kit to determine the viability of the bacteria before every experiment. Both B. henselae and B. vinsonii were tested on the day of assay before antibiotic treatment (considered as time point 0 hours). Viability was also measured after 96 hours on control, untreated bacteria. On day-1 of the assay, the viable bacteria were around 97% compared to 73% after 96 hours. The decrease in the viable bacteria after 96 hours could be due to the bacterial cells reaching stationary phase. Bacteria treated with therapeutic agents yielded inconclusive results.

As such, there were bacteria that showed only SYTO9 signal when treated with azithromycin, ceftriaxone, and azlocillin concentrations of 10 μg/ml, the concentration at which there was no growth on blood agar plates. For this reason, live/dead staining was not taken into consideration in the determination of MBC either in cell-free liquid culture assay or DH82 cell-based assay.

### Equivalents and Scope

Those skilled in the art will recognize, or be able to ascertain using no more than routine experimentation, many equivalents to the specific embodiments in accordance with the present disclosure. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

The term “about,” as used herein, refers to variation in the numerical quantity that can occur, for example, through typical measuring techniques and equipment, with respect to any quantifiable variable, including, but not limited to, mass, volume, time, distance, wave length, frequency, voltage, current, and electromagnetic field. Further, given solid and liquid handling procedures used in the real world, there is certain inadvertent error and variation that is likely through differences in the manufacture, source, or purity of the ingredients used to make the compositions or carry out the methods and the like. The term “about” also encompasses amounts that differ due to different equilibrium conditions for a composition resulting from a particular initial mixture. The term “about” also encompasses these variations. Whether or not modified by the term “about,” the claims include equivalents to the quantities.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A method of reducing intracellular levels of Bartonella in a population of cells, the method comprising:
contacting the population of cells with one or more therapeutic agents,
wherein the one or more therapeutic agent is selected from the group consisting of azlocillin, azithromycin, ceftriaxone, doxycycline, gentamicin and ampicillin; and
wherein contacting the population of cells with the one or more therapeutic agents reduces the intracellular levels of Bartonella in the population of cells.

2. The method of claim 1, wherein the one or more therapeutic agents comprises azlocillin.

3. The method of claim 1, wherein the method comprises contacting the population of cells with two therapeutic agents.

4. The method of claim 3, wherein the two therapeutic agents are azlocillin and azithromycin.

5. The method of claim 4, wherein the azlocillin is at a concentration of from about 0.5 μg/ml to about 16 μg/ml.

6. The method of claim 4, wherein the azithromycin is at a concentration of from about 0.5 g/ml to about 16 μg/ml.

7. (canceled)

8. (canceled)

9. A method of killing or reducing the growth of Bartonella in a subject, the method comprising:
administering to the subject in need thereof one or more therapeutic agents,
wherein the one or more therapeutic agents is azlocillin, azithromycin, ceftriaxone, doxycycline, gentamicin, or ampicillin; and
wherein the administering to the subject in need thereof with the one or more therapeutic agents kills and/or inhibits the growth of the Bartonella.

10. The method of claim 9, wherein the one or more therapeutic agents is at a concentration of from about 0.001 μg/ml to about 5 μg/ml.

11. The method of claim 9, wherein the one or more therapeutic agents is azlocillin.

12. The method of claim 11, wherein the azlocillin is at a concentration of from about 0.001 μg/ml to about 2 μg/ml.

13. (canceled)

14. A method of treating Bartonellosis in a subject, the method comprising:
administering to the subject in need thereof one or more therapeutic agents,
wherein the one or more therapeutic agents is selected from the group consisting of azlocillin, azithromycin, ceftriaxone, doxycycline, gentamicin, and ampicillin; and
wherein the administering to the subject in need thereof with the one or more therapeutic agents reduces one or more symptoms associated with Bartonellosis.

15. The method of claim 14, wherein the Bartonellosis in the subject is caused by Bartonella bacilliformis, B. quintana, B. henselae, B. elizabethae, B. vinsonii, B. clarridgeiae, B. grahamii, B. koehlerae, B. alsatica, B. rochalimae, B. phoceensis, B. rattimassiliensis, B. tamiae, B. washoensis, or B. australis.

16. The method of claim 14, wherein the Bartonellosis is Cat Scratch Disease, Trench Fever, or Carrion's disease.

17. The method of claim 14, wherein the one or more therapeutic agents are independently at a concentration of from about 0.001 μg/ml to about 5 μg/ml.

18. The method of claim 14, wherein the method comprises administering two therapeutic agents to the subject in need thereof.

19. The method of claim 18, wherein the two therapeutic agents are azlocillin and azithromycin.

20. The method of claim 19, wherein the azlocillin is at a concentration of from about 0.5 μg/ml to about 16 μg/ml.

21. (canceled)

22. The method of claim 19, wherein the azithromycin is at a concentration of from about 0.5 μg/ml to about 16 μg/ml.

23. (canceled)

24. The method of claim 14, wherein the one or more therapeutic agent is azlocillin.

25. The method of claim 24, wherein the azlocillin is at a concentration of from about 0.001 μg/ml to about 2 μg/ml.

26. (canceled)
